(19) United States
(12) Patent Application Publication
(10) Pub. No.: US 2009/0012038 A1
(43) Pub. Date: Jan. 8, 2009

(54) Methods for microbial biofilm destruction and interference with microbial cellular physiology

(76) Inventors: Alfred M. Spormann, Stanford, CA (US); Kai M. Thormann, Bochum (DE); Renee M. Saville, Mountain View, CA (US)

Correspondence Address: Bozicevic, Field & Francis LLP;Stanford University Office of Technology Licensing, 1900 University Avenue, Suite 200, East Palo Alto, CA, 94303, US

(21) Appl. No.: 11/665,418

(22) PCT Filed: Oct. 18, 2005
(86) PCT No.: PCT/US05/37880

§ 371 Date: Feb. 21, 2008

Related U.S. Application Data
(60) Provisional application No. 60/619,973, filed on Oct. 18, 2004.

(51) Int. Cl. A61K 31/7076; C12Q 1/02; A61P 31/00

(52) U.S. Cl. 514/48; 435/29

(57) ABSTRACT

The formation and maintenance of microbial biofilms is shown to be dependent on signaling pathways mediated by cyclic di-GMP. In the absence of such signaling, microbes detach from a biofilm, and thereby become more readily treatable with conventional antibiotics. Chemical or biological means that interfere with cyclic-di-GMP signaling induce biofilm dissolution, providing for a new class of antibiotics. In one embodiment of the invention, the biofilm inhibitor is an analog of cyclic-di-GMP, which competitively or non-competitively blocks signaling. In another embodiment of the invention, the biofilm inhibitor is a genetic sequence that interferes with cyclic-di-GMP synthesis or signaling.

A biofilm is an accumulation of microorganisms (bacteria, fungi, and/or protozoa, with associated bacteriophages and other viruses) embedded in a polysaccharide matrix and adherent to solid biological or non-biotic surfaces. Biofilms are medically important, accounting for over 80 percent of hospital-acquired microbial infections in the body. Examples include infections of the: oral soft tissues, teeth and dental implants; middle ear; gastrointestinal tract; urogenital tract; airway/lung tissue; eye; urinary tract prostheses; peritoneal membrane and peritoneal dialysis catheters, indwelling catheters for hemodialysis and for chronic administration of chemotherapeutic agents (Hickman catheters); cardiac implants such as pacemakers, prosthetic heart valves, ventricular assist devices, and synthetic vascular grafts and stents; prostheses, internal fixation devices, percutaneous sutures; and tracheal and ventilator tubing. The microorganisms tend to be far more resistant to antimicrobial agents and to be particularly difficult for the host immune system to render an appropriate response.

Biofilms are remarkably difficult to treat with antimicrobials. Antimicrobials may be readily inactivated or fail to penetrate into the biofilm. In addition, bacteria within biofilms have increased (up to 1000-fold higher) resistance to antimicrobial compounds, even though these same bacteria are sensitive to these agents if grown under planktonic conditions.

Biofilms increase the opportunity for gene transfer between/among bacteria. This is important since bacteria resistant to antimicrobials or chemical biocides can transfer the genes for resistance to neighboring susceptible bacteria. Conjugation occurs at a greater rate between cells in biofilms than between planktonic cells. The probable reason for enhanced conjugation is that the biofilm environment provides minimal shear and closer cell-to-cell contact. Since plasmids may encode for resistance to multiple antimicrobial agents, biofilm association also provides a mechanism for selecting for, and promoting the spread of, bacterial resistance to antimicrobial agents. Gene transfer can convert a previous avirulent commensal organism into a highly virulent pathogen.

Certain species of bacteria communicate with each other within the biofilm. As their density increases, the organisms secrete low molecular weight molecules that signal when the population has reached a critical threshold. This process, called quorum sensing, is responsible for the expression of virulence factors. For example, two different cell-to-cell signaling systems in P. aeruginosa, lasR-lasI and rhlR-rhlI, are involved in biofilm formation. At sufficient population densities, these signals reach concentrations required for activation of genes involved in biofilm differentiation. Mutants unable to produce both signals (double mutant) were able to produce a biofilm, but unlike the wild type, their biofilms were much thinner, cells were more densely packed, and the typical biofilm architecture was lacking. In addition, these mutant biofilms were much more easily removed from surfaces by a surfactant treatment. Induction of genetic competence (enabling the uptake and incorporation of exogenous DNA by transformation) is also mediated by quorum sensing in S. mutans. Transformational frequencies were 10-600 times higher in biofilms than planktonic cells.

Several frank bacterial pathogens have been shown to associate with, and in some cases, grow in biofilms, including Legionella pneumophila, S. aureus, Listeria monocytogenes, Campylobacter spp., E. coli O157:H7, Salmonella typhimurium, Vibrio cholerae, and Helicobacter pylori. Although all these organisms have the ability to attach to surfaces and existing biofilms, most if not all appear incapable of extensive growth in the biofilm. This may be because of their fastidious growth requirements or because of their inability to compete with indigenous organisms. Survival and growth of pathogenic organisms within biofilms might also be enhanced by the association and metabolic interactions with indigenous organisms.

Bacteria embedded within biofilms are resistant to both immunological and non-specific defense mechanisms of the body. Contact with a solid surface triggers the expression of a panel of bacterial enzymes, which catalyze the formation of sticky polysaccharides that promote colonization and protection. The structure of biofilms is such that immune responses may be directed only at those antigens found on the outer surface of the biofilm, and antibodies and other serum or salivary proteins often fail to penetrate into the biofilm. In addition, phagocytes are unable to effectively engulf a bacterium growing within a complex polysaccharide matrix attached to a solid surface. This causes the phagocyte to release large amounts of pro-inflammatory enzymes and cytokines, leading to inflammation and destruction of nearby tissues.

Biofilm cells may be dispersed either by shedding of daughter cells from actively growing cells. In as much formation of a biofilm comprises a critical lifestyle decision for a microbe with significant implications for the surrounding human or natural environment, the detachment or dissolution of a biofilm has fundamental consequences for the microbe and its environment. Although biofilm disintegration is observed frequently and considered to be part of a developmental biofilm program, the processes have been poorly understood.

Detachment is the transition of microbial cells from the biofilm to the planktonic state and is controlled by environmental or internal cues. Conceptually, the steps involved in detachment include at least three elements: i) sensing of environmental conditions, ii) signal relay, and iii) a molecular machinery mediating the separation of cells from the biofilm, the equivalent of activation of a ‘detachase’. The nature of the bonds that retain cells in a biofilm may play a critical role in detachment, which could be attributed to the breaking of such bonds between cells and the extracellular matrix (e.g., exopolysaccharide, including alginate, colanic acid and cellulose, DNA, proteins) or other cells (e.g., by fimbriae or pili) created during attachment.

In view of the importance of biofilms for microbial infection, methods of understanding and manipulating biofilm dissolution are of great interest, and are addressed herein.

### SUMMARY OF THE INVENTION

Compositions and methods are provided for the dissolution of microbial biofilms. It is shown herein that the formation and maintenance of such biofilms is dependent on signaling pathways mediated by cyclic di-GMP. In the absence of such signaling, microbes detach from a biofilm, and thereby become more readily treatable with conventional antibiotics.

Chemical or biological means that interfere with cyclic-di-GMP signaling induce biofilm dissolution, providing targets for a new class of antibiotics. In one embodiment of the invention, the biofilm inhibitor is an analog of cyclic-di-GMP, which competitively or non-competitively blocks signaling. In another embodiment of the invention, the biofilm inhibitor is a genetic sequence that interferes with cyclic-di-GMP synthesis or signaling.

A pharmaceutical composition comprising a biofilm inhibitor as an active agent is administered to a patient suffering from a microbial infection, particularly bacterial infections forming or derived from biofilms. The biofilm inhibitor may be administered alone, or in combination with other bacteriocidal agents, e.g. antibiotics, etc. Formulations may also find use in the in vitro dissolution of biofilms, e.g. in hospital settings, and may be combined with other bacteriocidal agents.

In another embodiment of the invention, methods of screening for biofilm inhibitors are provided, where candidate agents are screened for the ability to interact with cyclic-di-GMP signaling pathways; to increase the dissolution of bacterial films in test conditions, and the like.

In another embodiment of the invention, methods developed for chemical and biological interference with cyclic-di-GMP signaling are applied to interfere with infections by microbes not present in biofilms, by affecting the cellular metabolism and physiology, including interference with pathogenesis mechanisms.

### BRIEF DESCRIPTION OF THE DRAWINGS

FIG. 1. Organization of the mxdA-D genes in S. oneidensis MR-1. Horizontal arrows indicate the position of primers used in transcription analysis, vertical arrows mark the position of transposon insertions that led to the identification of the gene cluster. The mxdA-D genes correspond to the TIGR SO gene annotations SO4180-4177.

FIG. 2. Involvement of the mxdA-D genes in cell attachment and three-dimensional biofilm architecture. A) Biofilm phenotypes of (a) AS93 (wild type control), (b) ΔmxdA, (c) ΔmxdB, and (d) ΔmxdC in hydrodynamic flow chambers. Images display shadow projections of biofilms formed after 48 h. x-z and y-z cross-sectional images at selected positions in the biofilm are shown at the bottom and right side, respectively. The scale bar represents 70 μm. B) Quantification of biomass of the ΔmxdA-D mutants. CLSM images of the experiments shown in A) were quantified by COMSTAT. C) Complementation of ΔmxdA and ΔmxdB by GGDEF-encoding VCA0956. Biofilm formation in 96-well microtiter plates was measured by crystal violet staining of cells attached to the walls as described in the Materials and Methods section.

FIG. 3. Intracellular content of c-di-GMP. C-di-GMP content in planktonically-grown cells were determined as described in Material and Methods, and are expressed per mg wet weight cells. The strains assayed included AS145 (AS93+pAra::yhjH), AS146 (AS93+pAra::VCA0956), AS147 (AS93+empty vector), AS152 (AS140+pLacTac::mxdA), and AS160 (AS140+empty vector).

FIG. 4. Truncation analysis of MxdA. 5′ and 3′ deletions were introduced in mxdA and strains expressing these alleles in a ΔmxdA genetic background were tested for biofilm formation by rescuing the ΔmxdA phenotype. Rectangles on the left indicate the size and region of the expressed protein relative to wild type; bars on the right indicate biofilm biomass formed in a 96-well plate assay. The MxdA NVDEF region spans amino acids at positions 357-361.

FIG. 5. Effects of VCA0956 and yhjH expression on S. oneidensis biofilm architecture. Images display shadow projections of biofilms formed after 24 h of (a) AS147, (b) AS146 constitutively over-expressing VCA0956 and (c) AS145 constitutively over-expressing yhjH. The inocula were grown in LB containing 25 μg/ml kanamycin, diluted to an OD600 of 0.01 and injected into the flow chamber. After 40 minutes, the flow of LM containing 25 μg/ml kanamycin and 0.2% arabinose as inducer was initiated. The scale bar represents 200 μm.

FIG. 6. Effects of VCA0956 and yhjH expression on developed S. oneidensis wild type biofilms. Displayed are the amounts of biomass detached relative to the detached biomass of wild type AS147 (set to 100%). Biofilms of strains AS147, AS145, and AS146 were grown in flow chambers for 20 hours prior to induction with 0.2% (w/v) L-arabinose. After 90 minutes of induction detachment was induced by a stop-of-flow (grey shaded bars). The white bar indicates biomass detached from AS145 after 120 min without a stop-of-flow.

FIG. 7. Model for control of attachment and detachment by c-di-GMP in S. oneidensis. An environmental cue is sensed by sensor protein(s), which modulate the enzymatic activity of c-di-GMP-forming diguanylate cyclases(s), such as GGDEF domain containing proteins and MxdA, and/or of c-di-GMP-hydrolyzing phosphodiesterase(s), such as EAL domain-containing proteins. An altered general or localized c-di-GMP pool allosterically affects the activity of proteins or enzymes involved in attachment and/or detachment, such as MxdB. MxdA is postulated to be a key c-di-GMP-forming enzyme and to function in (structural) context with the putative glycosyl transferase MxdB. The c-di-GMP level (of a general intracellular or a localized pool) can be controlled in several ways: a) via activation/inhibition of diguanylate cyclase(s) or by inhibition/activation of phosphodiesterases. Upon activation, MxdA catalyzes the formation of c-di-GMP, which stimulates the polysaccharide synthesis activity of MxdB, resulting in increased attachment. Downregulation of MxdA or activation of phosphodiesterase(s), and subsequent decreased glycosyl transferase activity could result in decreased attachment (i.e. detachment). At this point it is unclear whether detachment is due to the activation of a ‘detachase’ or the inhibition of an attachment activity such as MxdB by low c-di-GMP.

### DETAILED DESCRIPTION OF THE EMBODIMENTS

Methods are provided for the use of biofilm inhibitors as antimicrobial agents. Biofilm inhibitor(s) are administered alone or in combination with other active agents to a patient suffering from or predisposed to an infection comprising biofilm formation, in a dose and for a period of time sufficient to reduce the patient population of microbial pathogens. It is also found that methods of interfering with c-di-GMP affect other aspects of microbial metabolism and physiology, including pathogenesis mechanisms.

There is a continuing need for new antimicrobial agents, particularly those that are effective in killing pathogens resistant to conventional antibiotics. Specific treatments of interest include, without limitation, treatment of patients having implantable medical devices.

A biofilm is an assemblage of microbial cells that is closely associated with a surface and enclosed in a matrix of material, including polysaccharides, DNA, and proteins. Noncellular materials such as mineral crystals, corrosion particles, clay or silt particles, or blood components, depending on the environment in which the biofilm has developed, may also be found in the biofilm matrix. Biofilm-associated organisms also differ from their planktonic (freely suspended) counterparts with respect to the genes that are transcribed. Biofilms may form on a wide variety of surfaces, including living tissues, indwelling medical devices, industrial or potable water system piping, or natural aquatic systems.

The solid-liquid interface between a surface and an aqueous medium provides an ideal environment for the attachment and growth of microorganisms. The solid surface may have several characteristics that are important in the attachment process. The extent of microbial colonization appears to increase as the surface roughness increases. This is because shear forces are diminished, and surface area is higher on rougher surfaces. The physicochemical properties of the surface may also exert a strong influence on the rate and extent of attachment. Microorganisms attach more rapidly to hydrophobic, nonpolar surfaces such as Teflon and other plastics than to hydrophilic materials such as glass or metals.

Other characteristics of the aqueous medium, such as pH, nutrient levels, ionic strength, and temperature, may play a role in the rate of microbial attachment to a substratum. Several studies have shown a seasonal effect on bacterial attachment and biofilm formation in different aqueous systems. This effect may be due to water temperature or to other unmeasured, seasonally affected parameters.

Cell surface hydrophobicity, presence of fimbriae and flagella, and production of EPS all influence the rate and extent of attachment of microbial cells. The hydrophobicity of the cell surface is important in adhesion because hydrophobic interactions tend to increase with an increasing nonpolar nature of one or both surfaces involved (i.e., the microbial cell surface and the substratum surface). Most bacteria are negatively charged but still contain hydrophobic surface components. Fimbriae, i.e., nonflagellar appendages other than those involved in transfer of viral or bacterial nucleic acids, contribute to cell surface hydrophobicity. Most fimbriae that have been examined contain a high proportion of hydrophobic amino acid residues. Fimbriae play a role in cell surface hydrophobicity and attachment, probably by overcoming the initial electrostatic repulsion barrier that exists between the cell and substratum. A number of aquatic bacteria possess fimbriae, which have also been shown to be involved in bacterial attachment to animal cells.

Other cell surface properties may also facilitate attachment. Several studies have shown that treatment of adsorbed cells with proteolytic enzymes caused a marked release of attached bacteria, providing evidence for the role of proteins in attachment. The O antigen component of lipopolysaccharide (LPS) has also been shown to confer hydrophilic properties to gram-negative bacteria.

Without being bound by theory, it is believed that the mechanism of detachment from a biofilm is as follows. Detachment occurs in rapid response to a sudden decrease of essential nutrients, such as O₂ concentration. The metabolic state of biofilm cells is critical for detachment. Restricting, e.g. oxygen transport into the biofilm by stop-of-flow results in rapid O₂ depletion in the biofilm because of the rapid cellular oxygen consumption by obligatorily respiring cells. This, in conjunction with the phenotypes of the global redox regulator mutant s (such as CRP, ArcA, and EtrA), points to O₂ metabolism as a critical parameter in detachment. Under standard biofilm growth conditions, it is believed that a slow, metabolically-driven decrease in local oxygen concentration leads to an adaptation of subpopulations, particularly in the deeper layers of biofilms, to the new conditions of oxygen limitation, and only minor detachment from the biofilm occurs while most of the cells stay within the community.

Populations, especially in the internal layers of thicker e.g. >18 h biofilms, have presumably been adapting gradually to a reduced metabolic activity due to low oxygen concentration as the biofilm increases in thickness. As demonstrated for P. putida, the metabolically more active cells are those that are in direct contact with the medium flow. The existence of cell layers in S. oneidensis biofilms with lower internal metabolic activity is supported by the observation that GFP fluorescence increased in lower biofilm layers after the top layers were removed by induced detachment. Once such internal cell layers are exposed to higher oxygen concentration, for example after detachment of the top cell layers, their metabolic activity is expected to increase, thereby adapting to a metabolism at elevated oxygen concentration. If these cells then were rapidly deprived of oxygen, e.g. by another stop of flow, the cells of this layer would regain the ability to respond to the sudden decrease in oxygen by detachment. Therefore, the absence of an apparent threshold for oxygen concentration and for biofilm thickness together with the restoration of detachment-competence suggest that a sudden oxygen decrease, which is more rapid than a metabolic adaptation, is the direct trigger inducing detachment in metabolically active cells such as in S. oneidensis.

Mdx operon. MxdA is a 462 amino acid protein containing a C-terminal region with weak homology to a GGDEF domain. MxdB is a membrane-associated 403 amino acid protein with homology to glycosyl transferases of the family GT 2 type. MxdC is a 351 amino acid membrane-associated protein with homology to efflux pump proteins. MxdD is oriented in the same direction as the previous genes, and is a 118 amino acid membrane-associated protein without homology to any known protein. The MxdB amino acid sequence was 25% identical and 42% similar to that of AcsAB, the cellulose synthase of G. xylinus, over a range of 192 amino acids. The orientation and sequences of the mxd genes are highly similar and homologous, respectively, only to the Vibrio parahaemolyticus RIMD genes VPA0392-94.

In one embodiment of the invention, screening assays utilize a peptide derived from the mxdA-D operon. A polypeptide of particular interest is the mdxA polypeptide or a fragment derived therefrom. Fragments of interest include at least about 12 amino acids, at least about 16 amino acids, at least about 20 amino acids, at least about 25 amino acids, at least about 50 amino acids, at least about 100 amino acids, at least about 200 amino acids, and may comprise a contiguous sequence derived from mdxA. Such peptides preferably include the NVDEF sequence at positions 357-361. In complementation assays it has been found that expression of the NVDEF domain alone was sufficient to partially confer rescue. MxdA can function as a diguanylate cyclase with an essential, modified GGDEF-like NVDEF domain.

The complete genome sequence of Shewanella oneidensis MR-1 is publicly available, for example at Genbank, accession NC_{—}004347. The sequence of the genes in the operon are as follows, where reference is made to the genome sequence:

MdxD, “SO4177”, coding sequence, complement (4349450 . . . 4349806)

MTQSAIISILLCIAVLLGAMYFGSWTKHPYVQELTTEICKASAVIEPKKPSAEVMQEDKS KAESSSLPKVIWDQMLDNTSPPKPPVSPLFNQSLPRQEDSATTPDHSGADETRVEPLE

MdxC, “SO4178”, coding sequence, complement (4349988 . . . 4351043)

MKVNYQPSHKAQRPTDDKGIGVKYAAAKRGGFKGRWYLLLTLVIAPVVVVGWILLRP HLFILASGIVTTEPLEVRAPSAGDVAAIMVKRGDVLASGANILTLVDTQLGAQIQELEKQLS QLEFDHLSLNAEILTQLQQRIAVAAEGVTRQDGLLDSFERYQRQGVVPTADMAAVLQAHT ASKMALEQAKVDLMQARQGQKTELLAGAIAQSKYNIELQLARLKAQESQLHIKALNPTRV VDVLVQVGEHIVEDRPLVLLSGREAAVIFAYLEPKYLEYTNIGQKATIKLPNSTWLRAEISE PTELVGRLPKQLSGPFDGEKPVLKITLKPETALPTAIEGVPVEVSFDYLW

MdxB, “SO4179”, coding sequence, complement (4351043 . . . 4352254)

MLLVLTGIMRFYYTSFVQIPRQSLYKPKVSCVITCYAEGEAVKSTIDSFIEQVYDGEIEII AVVDGAVQNALTYQVAMAAAKACQVPNRKVVVLPKWQRGGRVSTLNAGLSVATGEIVIN ADADTSFDNDMVSQIVPYFEDPNVPAVGGALRVRNVNESILTRMQAIEYLISMQGGKTGL GQWNLLNNISGAFGAFRRTFLIQIGGWDTHTAEDLDLTVRIKQYFKRHPDWHIPFATLAV GHTDAPADLKTLVLQRLRWDGDLLFLYFRKHWPAFTPKLLGTGTFLFTLLYGFLQNVLMP FVIVFYSLGIVLSYPWQFITSISLTIYGFYLAILVFFYLVVLLAISERLSQDLRLAIWLPLYPFY ALFMRLVCLFALLNEVVRRSHEESSMAPWWVLKRGRKF”

MdxA, “SO4180”, coding sequence complement (4352368 . . . 4353756)

MTSKLSLNAINNCYLDNFVWIGPCHQHPWHPEFETFNSVEALLEQGRTVELIVLSLQA EEQDKCLRALRKNDSTFLSHILVCHESALSPYLANGLWDAGYDECYQIYQLKKKQIKLDY HDDPRYKLLTYLWCHENTILEPHSVPEKTYLYDYPLLRCFGINPEESFAWLGELQKSQLIE KAELSNRLRFCPSCHSGHLNYIDVCPQCHSIDTELQSSLHCFNCGHVGAQASFRKLNTLS CPNCLQSLRHIGVDYDRPIENQHCNSCQTLFVDAVVEAKCLHCQVSSKLNDLHVRNVYS FKLAITGRTLVRQGRSLSWFALEPGEQMTSAQFYWLLDWQNKLAKRHHQTHSILSIQML NVDEFLRAEGEAKGFAQLDALQDRLRSVIRVTDACSNYTRDGLLMLLPMTEMSQLNSIYK KLFDLKELQSTSKIEFSVKALTLPAEIGENVAEWLTDQLVKAKPI

Screening Assays: Candidate agents may be screened for their ability to interfere with cyclic-di-GMP signaling. Inhibitors find use in the dissolution of biofilms, and in the interference of other pathways mediated by c-di-GMP. Assays to determine affinity and specificity of binding are known in the art, including competitive and non-competitive assays. Assays of interest include ELISA, RIA, flow cytometry, 96-well-based biofilm screens, etc. In one such example, biofilms of tester bacteria are grown in 96-well plates where they form biofilms on the plastic surface. Then, test compounds are added and the loss of biomass from the wells is quantified. Binding assays may use mdx polypeptides as described above, may use purified or semi-purified GGDEF or mdxA domain proteins, or alternatively may use cells that express such GGDEF or mdxA domain proteins; cells transfected with an expression construct for such GGDEF or mdxA domain proteins, etc.

Generally a plurality of assay mixtures are run in parallel with different agent concentrations to obtain a differential response to the various concentrations. Typically, one of these concentrations serves as a negative control, i.e. at zero concentration or below the level of detection. The results in the absence (control) and presence of the agent are compared.

Conveniently, in these assays one or more of the molecules will be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule, which provides for detection, in accordance with known procedures.

In vitro binding assays may be provided in a wide variety of materials and shapes e.g. microtiter plate, microbead, dipstick, resin particle, etc. The substrate is chosen to minimize background and maximize signal to noise ratio. Binding may be quantitated by a variety of methods known in the art. After an incubation period sufficient to allow the binding to reach equilibrium, the insoluble support is washed, and the remaining label quantitated. Agents that interfere with binding will decrease the detected label.

Candidate agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification, halogenation to produce structural analogs.

Candidate agents also include genetic agents that interfere with expression of an mdx operon polypeptide, e.g. mdxA. Such agents include genetic sequences encoding dominant negative mutants; antisense polynucleotides, RNAi polynucleotides, and the like as known in the art. Homologs of the mdx operon, e.g. mdxA, are readily identified by those of skill in the art. For example, such polypeptides may have at least about 25% sequence identity at the amino acid level, at least about 40% sequence identity at the amino acid level, at least about 50% sequence identity at the amino acid level, at least about 70% sequence identity at the amino acid level, at least about 80% sequence identity at the amino acid level, at least about 90% sequence identity at the amino acid level, or more. The presence of a GGDEF or NVDEF is indicative of a similar functional domain.

A variety of other reagents may be included in the screening assay. These include reagents like salts, neutral proteins, e.g. albumin, detergents, etc which may be used to facilitate optimal protein-DNA binding and/or reduce non-specific or background interactions. Also reagents that otherwise improve the efficiency of the assay, such as protease inhibitors, nuclease inhibitors, anti-microbial agents, etc. may be used.

Functional assays of interest include an assessment of the functional dissolution of microbial biofilms, e.g. as set forth in the examples. A candidate agent may be added to a biofilm under conditions where O₂ or cyclic-di-GMP signaling has not been initiated, and the release of microbes from the film quantitated.

In some embodiments of the invention, the biofilm inhibitor is an analog of cyclic-di-GMP. Such analogs may have the general structure as follows:

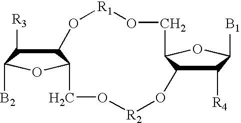

where B₁ and B₂ may be the same or different, and are independently selected from nitrogenous bases and derivatives thereof, including guanosine, inosine, adenine, guanine derivatives modified at the 6-position with S, N and O heteroatoms, 6-thioguanine, 2,6-diaminopurine, O6-alkyl guanine derivatives; and the like.

R₁ and R₂ may be the same or different, and are independently selected from PO₂; C═O; O—P═S (phosphorothioate); S═P═S (phosphorodithioate); O═P—BH₃ (boranophosphates); phosohoroamidite; O═P—CH₃ (methyl phosphonate); methane phosphonamidite; amide; methylene(methylimino); thioformacetal; dimethylene sulfone; sulfonamide; sulfonate; 5′N-sulfamate; sulfamide; 3′N-sulfamate; replacement of the entire phosphodiester with a guanidium or morpholino group; and the like.

R₃ and R₄ may be the same or different, and are independently selected from H, hydroxyl, ethers of lower alkyls; esters; CO₂H; thiols; phosphates, boronates lower alkyls, including methyl, ethyl, propyl, butyl, t-butyl, etc.

Formulations of biofilm inhibitors are administered to a host suffering from or predisposed to a microbial infection. Administration may be topical, localized or systemic, depending on the specific microorganism, preferably it will be localized. Generally the dose of biofilm inhibitor will be sufficient to decrease the microbial population in the biofilm by at least about 50%, usually by at least 1 log, and may be by 2 or more logs of release. The compounds of the present invention are administered at a dosage that reduces the microbial population while minimizing any side-effects. It is contemplated that the composition will be obtained and used under the guidance of a physician for in vivo use.

Biofilm inhibitors are also useful for in vitro formulations to dissolve microbial biofilms. For example, biofilm inhibitors may be added to hospital equipment, e.g. ventilation, water processing, etc.

The susceptibility of a particular microbe to biofilm inhibitors may be determined by in vitro testing, as detailed in the experimental section. Typically a culture of the microbe in the biofilm is combined with inhibitors at varying concentrations for a period of time sufficient to allow the protein to act, usually between about one hour and one day. The attached microbes are then counted, and the level of dissolution determined.

Microbes of interest, include, but are not limited to: Citrobacter sp.; Entembacter sp.; Escherichia sp., e.g. E. coli; Klebsiella sp.; Morganella sp.; Proteus sp.; Providencia sp.; Salmonella sp., e.g. S. typhi, S. typhimurium; Serratia sp.; Shigella sp.; Pseudomonas sp., e.g. P. aeruginosa; Yersinia sp., e.g. Y. pestis, Y. pseudotuberculosis, Y enterocolitica; Franciscella sp.; Pasturella sp.; Vibrio sp., e.g. V. cholerae, V. parahemolyticus; Campylobacter sp., e.g. C. jejuni; Haemophilus sp., e.g. H. influenzae, H. ducreyi; Bordetella sp., e.g. B. pertussis, B. bronchiseptica, B. parapertussis; Brucella sp., Neisseria sp., e.g. N. gononrhoeae, N. meningitidis, etc. Other bacteria of interest include Legionella sp., e.g. L. pneumophila; Listeria sp., e.g. L. monocytogenes; Mycoplasma sp., e.g. M. hominis, M. pneumoniae; Mycobacterium sp., e.g. M. tuberculosis, M. leprae; Treponema sp., e.g. T. pallidum; Borelia sp., e.g. B. burgdorfen; Leptospirae sp.; Rickettsia sp., e.g. R. rickettsii, R. typhi; Chlamydia sp., e.g. C. trachomatis, C. pneumoniae, C. psiffaci; Helicobacter sp., e.g. H. pylori, Staphylococcus sp., Streptococci sp. etc.

Non bacterial pathogens of interest include fungal and protozoan pathogens, e.g. Plasmodia sp., e.g. P. falciparum, Trypanosoma sp., e.g. T. brucei; shistosomes; Entaemoeba sp., Cryptococcus sp., Candida sp, e.g. C. albicans; etc.

Various methods for administration may be employed. The formulation may be given orally, or may be injected intravascularly, subcutaneously, peritoneally, by aerosol, opthalmically, intra-bladder, topically, etc. For example, methods of administration by inhalation are well-known in the art. The dosage of the therapeutic formulation will vary widely, depending on the specific biofilm inhibitor to be administered, the nature of the disease, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered once or several times daily, semi-weekly, etc. to maintain an effective dosage level. In many cases, oral administration will require a higher dose than if administered intravenously.

### Formulations

The compounds of this invention can be incorporated into a variety of formulations for therapeutic administration. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, creams, foams, solutions, suppositories, injections, inhalants, gels, microspheres, lotions, and aerosols. As such, administration of the compounds can be achieved in various ways, including oral, buccal, rectal, parenteral, intraperitoneal, intradermal, transdermal, intracheal, etc., administration. The biofilm inhibitors may be systemic after administration or may be localized by the use of an implant or other formulation that acts to retain the active dose at the site of implantation.

The compounds of the present invention can be administered alone, in combination with each other, or they can be used in combination with other known compounds (e.g., antibiotics, etc.) In pharmaceutical dosage forms, the compounds may be administered in the form of their pharmaceutically acceptable salts. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

The compounds can be used as lotions, for example to prevent infection of burns, by formulation with conventional additives such as solubilizers, isotonic agents, suspending agents, emulsifying agents, stabilizers and preservatives.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant containing biofilm inhibitors is placed in proximity to the site of infection, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the unit dosage forms of the present invention depend on the particular compound employed and the effect to be achieved, and the pharmacodynamics associated with the compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

For use in the subject methods, biofilm inhibitors may be formulated with other pharmaceutically active agents, particularly other antimicrobial agents. Other agents of interest include a wide variety of antibiotics, as known in the art. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc.

Anti-mycotic agents are also useful, including polyenes, e.g. amphotericin B, nystatin; 5-flucosyn; and azoles, e.g. miconazol, ketoconazol, itraconazol and fluconazol. Antituberculotic drugs include isoniazid, ethambutol, streptomycin and rifampin.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Regulation of Detachment of Shewanella oneidensis MR-1 Biofilms by Cyclic-di-GMP

A physiological and molecular analysis of detachment was undertaken in Shewanella oneidnesis, which is a facultative Fe(III)- and Mn(IV)-mineral reducing soil bacterium and plays a critical role in mineral dissolution, heavy metal (im)mobilization, and pollution degradation. In a hydrodynamic flow chamber system, detachment of individual S. oneidensis cells can be initiated by a rapid decrease in molecular oxygen, but not in electron donor concentration. Single cell resolution CLSM revealed that detachment occurs as the separation of individual cells and of cells in small groups throughout the entire layers in a thin biofilm. Such oxygen-dependent detachment can be simply induced by stopping the flow of an oxygen-limited medium in the hydrodynamic biofilm system.

On average, this detachment can lead to a 50-80% loss of the biofilm biomass. Notably, an only 5 minute stop-of-flow already releases up to 50% of the cell mass. Mutants defective in global transcriptional regulators CRP, ArcA, and EtrA, which are known in S. oneidensis and other γ-proteobacteria to regulate physiological processes in response to oxygen, were defective in the detachment response when induced by a stop-of-flow. The rapid detachment response suggests that the oxygen-starvation induced detachment is consistent with either an allosteric activation of an already present ‘detachase’, or an inhibition of a constitutive adhesion machinery, related to the c-di-GMP/exopolysaccharide biosynthesis system.

### Results

Transcription is not required for the oxygen starvation-induced detachment. To directly test whether transcription is required for detachment upon rapid decrease in molecular oxygen concentration, we grew biofilms of S. oneidensis in LM medium for 12 h, and subsequently switched the medium to a LM medium amended with 10 μg/ml rifampicin for 10 or 40 minutes, respectively. After that time period, the medium flow was stopped to induce detachment. Rifampicin-treated cells were found to be detached to the same extent as the untreated cells, indicating that transcription is not required.

In order to assure that rifampicin indeed inhibited transcription in biofilm cells under those conditions, we designed an experiment to test transcription in biofilm cells. Tester cells were S. oneidensis cells tagged with an IPTG inducible gfp under control of Pt, which is repressed by LacIQ. In the absence of IPTG, no GFP was produced in biofilms of strain AS129, and cells did not fluoresce. Upon addition of 10 mM ITPG to the flow-through-medium, the induction of GFP in biofilm cells was readily observed by CLSM throughout the entire biofilm and quantified. This assay is very sensitive, and after 1 hour, about 20% of the maximal inducible GFP fluorescence was observed. In contrast, biofilms treated with 10 μg/ml rifampicin did not yield any measurable fluorescence after 1 hour. These experiments show that rifampicin acts as a transcriptional inhibitor in our biofilms, and that this laIQ/IPTG system is a useful experimental tool to investigate transcription in biofilm cells. Furthermore, these transcriptional experiments show that transcription per se is not required for the O₂-dependent detachment response, demonstrating that the effect of the global transcriptional regulators CRP, ArcA, and EtrA is indirect.

Identification of an EPS operon in S. oneidensis. In order to identify the components involved in detachment, we focused on the components controlling cell adherence in S. oneidensis biofilms. Based on the S. oneidensis genome sequence, no genes encoding enzymes for exopolysaccharide (EPS) biosynthesis, similar as in Pseudomonas aeruginosa, E. coli, or Vibrio cholerae, are obvious. Previously, we conducted a genetic analysis using Tn5 mutagenesis coupled to a 96-well-based screen for biofilm-deficient mutants. In this screen, we isolated five transposon mutants that inserted in four independent positions of the gene cluster SO4180-4177; two insertions were in SO4180, one in the intergenic region between SO4180 and SO4179, and one insertion in SO4178.

Analysis of these open reading frames revealed that SO4180 is a 462 aa protein and contains a C-terminal domain with weak homology to a GGDEF motif. SO4179 is predicted to be a membrane-associated, 403 aa protein with homology to glycosyl transferases, SO 4178 is predicted to be a 351 aa protein. SO4177 is predicted to be a 118 aa protein and is oriented in the same direction as the previous genes.

In order to examine the function of theses genes in biofilm formation, we constructed in-frame deletion mutants of all genes, and examined their phenotype in biofilms grown under hydrodynamic conditions. Reconstruction of the null mutants is necessary as spontaneous insertion of IS sequences has been observed in S. oneidensis. All the mutants were found to have a severe defect in biofilm formation. In particular, deletions of SO4180, SO4179, and SO4178 were severely impacted in their ability to develop a three dimensional architecture.

The initial adhesion of these mutants appeared to be as in wild type, however the mutant biofilms arrested at the stage of a cell monolayer or few cell layers, and did not progress from this stage even after 72 h incubation. The most severe phenotype was visible in AS04180. Deletion mutant SO4177 showed a delayed phenotype but progressed to a wild type-like architecture after 48 h. These data together with the sequence analysis demonstrate that the gene cluster SO4180-4178 is essential for EPS biosynthesis in S. oneidensis.

The role of c-di-GMP in biofilm formation and detachment. The finding of a potential GGDEF protein (SO4180) in vicinity to a putative glycosyl transferase (SO4179) was intriguing. GGDEF domain-containing proteins are postulated to catalyze the synthesis of one mole cyclic-di-GMP from 2 moles GTP by a diguanylate cyclase activity. Such enzymatic activity has been shown for the GGDEF proteins VCA0956 of V. cholerae, AdrA of Salmonella typhimurium, and PleD of Caulobacter crescentus. Phosphodiesterase activity towards c-di-GMP has been shown for AxPDEA1 in Acetobacter xylinum, as well as for the EAL domain-containing protein YhjH (STM3611) of S. typhimurium. Cellulose synthase of A. xylinum was shown to be allosterically activated by c-di-GMP in biochemical experiments. Chang et al. reported that activity of the phosphodiesterase AxPDEA1 in Acetobacter xylinum is controlled by O₂. AxPDEA1 was shown to be a PAS domain-containing protein, which harbors a heme prosthetic group. Upon binding of molecular oxygen the phosphodiesterase activity is reduced three-fold, suggesting a link between oxygen and cellular c-di-GMP level.

In S. oneidensis the activity of an EPS synthase, such as SO04179, might be regulated by c-di-GMP, which is synthesized by GGDEF domain proteins, such as SO4180. This would imply that c-di-GMP is a signaling molecule coupling the sensing of rapid oxygen depletion with the rapid, transcription-independent detachment response. We, therefore, tested whether the V. cholerae protein VCA0956 with a known diguanylate cyclase activity could complement the biofilm phenotype of the ΔSO4180 bacteria.

The results demonstrated that SO4180 contains diguanylate cyclase activity and is involved in increasing the intracellular c-di-GMP level. The results further show that cellular c-di-GMP levels can control biofilm architecture, including detachment.

In order to test whether simple alterations of the intracellular c-di-GMP level per se and in the absence of a stop-of-flow affect detachment of S. oneidensis, we constructed two S. oneidensis strains. Strain AS1 carried the GGDEF domain-containing V. cholerae VCA0956, which was previously shown to carry diguanylate cyclase activity under the control of P_{tac} or Pₐᵣₐ and could be induced by addition of IPTG or arabinose. Similarly, strain AS2 carrying the EAL domain-containing YhjH, which contains phosphodiesterase activity under the control of P_{tac} or Pₐᵣₐ was constructed. Biofilms of both strains were grown in the absence of inducer. After 12 h of growth, the biofilm flow through medium was amended with 2 mM IPTG or arabinose to induce expression of the GGGDEF domain or EAL domain protein. CLSM images were taken and images were quantified.

The results demonstrate that the GGDEF domain expressing strain exhibited stronger attachment than wild type and a reduced detachment response. Conversely, the EAL-domain induced strain detached already rapidly in the absence of a stop-of-flow. These results strongly suggest that interfering with cellular c-di-GMP signaling by lowering the intracellular c-di-GP levels, here by overexpression of an EAL domain protein, induces rapid detachment and overrides cellular signaling.

External addition of c-di-GMP reversibly prevents biofilm detachment. The previous genetic experiments suggest that intracellular levels of c-di-GMP are critical for biofilm architecture. Specifically, they suggest that increased levels result in elevated biomass retention of a biofilm, whereas a decrease in intracellular c-di-GMP can result in detachment. We, therefore, predicted that amending the flow through medium of a biofilm in a hydrodynamic system with c-di-GMP should prevent the induction of detachment after applying a stop-of-flow. To test this hypothesis, biofilms of wild type S. oneidensis were grown in LM medium. After 12 h the medium was amended with 5 mM c-di-GMP and flow was continued. After 30 min, the flow was stopped to induce detachment and the non-detached biomass was quantified. It was found that no detachment was observed despite the stop-of-flow. In order to test whether the biofilm cells exposed previously to c-di-GMP were still competent for detachment after removal of c-di-GMP, the flow of medium was resumed, however, with LM medium devoid of c-di-GMP. Following an incubation of 2 h, the medium flow was stopped and the biofilm examined for detachment. It was found that under these conditions the biofilm still detached.

These results demonstrate that elevated concentration c-di-GMP prevent the detachment response upon stop-of-flow. Furthermore, these experiments demonstrate that external addition of c-di-GMP is accessible to cells and alters their physiology, thus, c-di-GMP or analogs thereof can provide a means of modulating biofilm formation.

The above data demonstrate that cellular levels of c-di-GMP respond to changes in molecular oxygen concentration. This is the first evidence that cellular c-di-GMP levels are metabolically controlled. Genetic and physiological evidence show that the rapid detachment of S. oneidensis cells from biofilms is controlled posttranscriptionally by c-di-GMP, and that at least one target of c-di-GMP regulation is a glycosyl transferase. The data furthermore imply that cellular c-di-GMP levels are metabolically controlled, presenting the first link of cellular c-di-GMP levels to environmental factors.

Interestingly, many environmental microbes contain a high fraction of GGDEF/EAL domain proteins. The presence of over 50 GGDEF/EAL domain proteins in the genome of S. oneidensis suggests that, similar to SO4180, environmental factors control the activity of these diguanylate cyclases.

Material & Methods
Strains & Plasmids
E. coli
S-17| λpir
DH5α λpir
K12-MG1655
S. oneidensis MR-1
MR-1
MR-1 Tn7-Plac-gfp (Renee, Soni)
AS93
-SO4180
-SO4179
-SO4178
-SO4177
+plasmids
plasmids
pGP704-Sac28-Km
pGP704-Sac28-Km-SO4180
pGP704-Sac28-Km-SO4179
pGP704-Sac28-Km-SO4178
pGP704-Sac28-Km-SO4177
pBAD44
(pMAL-c2)
pME6031
pME6041
pARA-gfp
pARA-VCA0956
pARA-yhjH
or
pLacTac-gfp
pLacTac-VCA0956
pLacTac-yhjH

Growth conditions & media. E. coli and Shewanella strains were grown in LB medium at 37 C and 30 C, respectively. When required, the medium was solidified with 1.5% (w/v) agar and supplemented with rifampicin, 10 μg/ml gentamicin, 20 μg/ml tetracycline, 25 μg/ml kanamycin, and 0.1% (w/v) L-arabinose (1 mM IPTG).

Strain construction in S. oneidensis MR-1. All genetic works were carried out according to standard protocols (Sambrook) or following the manufacturer's instructions. Kits for the isolation and/or purification of plasmid and chromosomal DNA or PCR fragments were obtained from Qiagen (Valencia, Calif.) and enzymes were purchased from New England Biolabs (Beverly, Mass.).

In-frame deletion mutants in S. oneidensis MR-1 AS93 were constructed essentially as reported earlier (Thormann et al.). Briefly, DNA fragments of the N- and C-terminal regions of the desired genes were amplified by PCR. The fragments were then digested with BamHI and SalI, respectively, and subsequently ligated. The resulting product was used in a second PCR amplification using the outer primers. The final product was purified, digested with NofI and SacI and ligated into the suicide-vector pGP704-Sac28-Km. The resulting plasmid was introduced into S. oneidensis MR-1 AS93 by mating from E. coli S-17l λpir, mutants yielded from single cross-over events were selected on LB plates containing kanamycin and gentamicin. To select for double crossover mutants, single colonies were grown overnight without antibiotic pressure and plated on LB containing 8% (w/v) sucrose. Kanamycin-sensitive mutants were then checked for the deletion by colony. PCR using primers up- and downstream of the deletion's location.

In order to construct an inducible system for S. oneidensis MR-1 based on the lacI^{q}-P_{tac} promoter system, a fragment containing the lacI^{q} gene and the tac promoter was amplified from the vector pMAL-c2, introducing BamHI and PstI restriction sites at the 5′- and 3′-end, respectively. Additionally, the −35 region of the lacI^{q} gene was mutated according to yield a higher expression of the repressor and, thus, a tighter repression of the system. The fragment was digested with BamHI and PstI and ligated into the broad host range-vector pME6041 that was treated with the same enzymes resulting in vector pLacTac. Genes to be cloned into this vector (gfp, yhjH, VCA0956) were amplified by PCR, introducing PstI and EcoRI restriction sites at the 5′ and 3′-ends, respectively.

Biofilm cultivation & Image acquisition. Cultivation of biofilms took place at 30° C. in three-channel flow cells with individual channel dimensions of 1×4×40 mm. As a substratum for microbial attachment microscope cover slips (Fisher Scientific, Pittsburgh, Pa.) were glued on with silicone (GE Sealants & Adhesives, Hunterville, N.C.) and left to dry for 24 h at room temperature prior to use. The assembly, sterilization, and inoculation of the flow system was carried out essentially as described (Thormann et al., 2004). Experiments were carried out in triplicates in at least two independent experiments.

For switching medium conditions, the flow was arrested and the medium was exchanged in the bubble trap and the upstream tubing. This process took no longer than 1 minute, control channels where the medium flow was stopped in parallel ensured that observed effects were not due to that short arrest. Confocal scans were taken at defined spots close to the inflow before and after indicated duration of the treatment.

Stop of flow-induced detachment was carried out as described earlier (Thormann). Briefly, the medium flow was arrested for 15 min and subsequentially resumed for the same amount of time. Pictures were taken immediately before the flow-stop and after 15 minutes of flow.

Microscopic visualization of and image acquisition from biofilms took place at the Stanford Biofilm Research Center using an upright Zeiss LSM510 Confocal Laser Scanning Microscope (Carl Zeiss, Jena, Germany) equipped with the following objectives: 10×/0.3 Plan-Neofluar, 20×/0.5W Achroplan, and 40×/1.2W C-Apochromat. For displaying biofilm images, CLSM images were processed using the IMARIS software package (Bitplane AG, Zürich, Switzerland) and Adobe Photoshop. Biofilm parameters, such as biomass, and average biofilm thickness were quantified with the program COMSTAT (Heydorn et al., 2000).

Biofilm cultivation and quantification in microtitre plates. Cultivation, staining, and quantification of S. oneidensis cells in microtitre plates was carried out as described earlier (Thormann et al., 2004). Cells were grown at 30 C in 175 μl LM medium for 16 hours prior to the staining procedure.

### Example 2

Most microbes in nature are assumed to exist as surface-associated communities in biofilms. Biofilms greatly affect their environment, be it the human host, a wastewater treatment plant, or pristine soils and sediments, and significant research has focused on understanding and controlling the resilience and stability of such biofilms. From a microbe's point of view, the decision to either remain associated with or to sever ties to and exit a biofilm confers profound consequences to its lifestyle. Stability and resilience of a three dimensional biofilm is controlled by two diametrically opposed states: attachment and detachment. These mutually exclusive states have in common a change in how cells are associated with the biofilm matrix. The biofilm matrix consists of exopolymeric substances, such as polysaccharides, DNA and proteins, but also of biofilm cells. Despite extensive research over the past decade on biofilms, adhesion of microbial cells to a substratum surface during initial contact, and the biofilm matrix, the molecular mechanisms of how biofilm cells stick to a biofilm and how such cells detach are largely unknown. For the purpose of this discussion, we define “adhesion” as the binding of a cell to a substratum, where as the term “attachment” is used to indicate the binding of a cell to a biofilm matrix.

Although biofilm disintegration is observed frequently and considered part of a developmental biofilm program, only recently systematic investigations provided some insights into the molecular events involved in detachment. Environmental cues, such as changes in oxygen or carbon substrate concentration, pH or other chemical parameters have been reported to induce detachment of mature biofilms. Consequently, detachment could be an active process where an environmentally controlled, direct activation of a ‘detachase’ initiates severing of bonds between cells and the biofilm matrix. Indeed, exopolysaccharide lyases and DNases have been implicated in cell dispersal from biofilms. Alternatively, detachment could also be a passive process. In this case, the opposite of detachment, i.e., attachment, could require constant (enzymatic) activity, and a sudden cessation of such ‘attachment activity’ could result in instant detachment. Finally, it is also conceivable that a combination of ‘attachment activity’ and of ‘detachase’ might be involved in physiologically controlled detachment.

Recently, quantitative, high-resolution confocal laser scanning microscopy in conjunction with a mutant analysis has provided some insights into the detachment process. Previously, we reported a genetic analysis of surface adhesion and biofilm formation, as well as a physiological and molecular characterization of detachment in Shewanella oneidensis MR-1, which is a facultative Fe(III)- and Mn(IV)-mineral reducing soil bacterium and plays a critical role in mineral dissolution, heavy metal (im)mobilization, and pollution degradation. In a genetic screen for biofilm defective mutants, we identified type IV pili as critical in mediating the initial adhesion of cells to the substratum during surface colonization. Furthermore, and similar to other biofilm microbes, flagellum motility was found to be important to control the architecture of biofilms. We also discovered that rapid detachment of individual S. oneidensis cells could be initiated by a sudden decrease in molecular oxygen, but not in electron donor concentration in a hydrodynamic flow chamber system. Single cell resolution CLSM revealed that detachment occurs as the separation of individual cells and of cells in small groups throughout all layers in a biofilm. Such oxygen-dependent detachment can be induced simply by stopping the flow of an aerobic, oxygen-limited growth medium in the hydrodynamic biofilm system. Notably, an only five-minute stop-of-flow releases up to 50% of the detachable cell mass. In this report, we identified a new, putative exopolysaccharide synthesis gene cluster that links the attachment and detachment of single cell to the biofilm matrix.

A molecular system, which was first discovered and studied in the control of extracellular cellulose biosynthesis in Gluconoacetobacter xylinus, formerly Acetobacterium xylinum, has been implicated in autoaggregation of planktonic cells and in biofilm formation in several microorganisms. In G. xylinus, the membrane-bound cellulose synthase is allosterically activated by the secondary messenger molecule, cyclic-di-GMP (cyclic-bis(3′,5′)guanylic acid). Cyclic-di-GMP (c-di-GMP) is synthesized by diguanylate cyclase and degraded by phosphodiesterase activities, respectively. Exopolysaccharide production relevant to biofilm formation may be allosterically controlled by c-di-GMP.

### Material and Methods

Growth conditions & media. E. coli strains were grown in Luria-Bertani (LB) medium at 37° C., Shewanella oneidensis MR-1 strains were grown at 30° C. in LB, Lactate Medium (LM,), or Mineral Medium (MM) of the following final composition: 485 μM CaCl₂×2H₂₅O; 5 μM CoCl₂; 0.2 μM CuSO₄×5H₂O; 57 μM H₃BO₃; 1.27 mM K₂HPO₄; 0.73 mM KH₂PO₄6; 1.0 mM MgSO₄×7H₂O; 1.3 μM MnSO₄; 67.2 μM Na₂EDTA; 3.9 μM Na₂MoO₄_{_{—}}2H₂7 O; 1.5 μM Na₂SeO₄; 150 mM NaCl; 2 mM NaHCO₃; 5 μM NiCl₂×5H₂O; 1 μM ZnSO₄8; 9 mM (NH₄)₂SO₄9; 15 mM lactate; 5 mM HEPES; pH 7.4, modified after). If required, the medium was solidified with 1.5% (w/v) agar and supplemented with 30 μg/ml chloramphenicol, 10 μg/ml gentamicin, 25 μg/ml kanamycin, and/or 20 μg/ml tetracycline. Gene induction from the pARA and pLacTac vectors was achieved by addition of 0.2% (w/v) L-arabinose or 1 mM isopropyl-D-D-thiogalactopyranosid (IPTG), if not indicated otherwise.

Strain constructions in S. oneidensis MR-1. All genetic work was carried out according to standard protocols or following the manufacturer's instructions. Kits for the isolation and/or purification of plasmid and chromosomal DNA or PCR fragments were obtained from Qiagen (Valencia, Calif.), and enzymes were purchased from New England Biolabs (Beverly, Mass.), if not indicated otherwise.

In-frame deletion mutants were constructed in S. oneidensis MR-1 AS93 essentially as reported. Briefly, DNA fragments of the N- and C-terminal regions of the selected genes were amplified by PCR, introducing suitable restriction sites. The fragments were digested with BamHI and SalI, respectively, and subsequently ligated. An aliquot of the ligation mixture was used as template in a second PCR amplification reaction using the forward primer of the upstream fragment and the reverse primer of the downstream fragment of the corresponding genes to generate the mutant allele. The products were purified, digested with NcoI and SacI, and ligated into the suicide-vector pGP704-Sac28-Km. The resulting plasmids (pGP704-Sac28-Km-mxdA to -mxdD) were introduced into S. oneidensis MR-1 AS93 by mating with E. coli S-171 Δpir, and mutants generated bysingle cross-over events were selected on LB plates containing kanamycin and gentamicin.

To select for double recombinants, single colonies were grown overnight in the absence of antibiotics, and plated on LB containing, 8% (w/v) sucrose. Kanamycin sensitive mutants were then screened for the gene deletion by colony PCR using primers up- and downstream of the deletion's location. The resulting mutants in mxdA-D were lacking the amino acids 25-447, 7-274, 111-379, and 35-107, respectively. In order to construct a system for controlled gene induction in S. oneidensis MR-1, genes of interest (gfp, VCA0956, yhjH) were amplified by PCR using DNA from the corresponding chromosomal DNA (Vibrio cholerae ElTor N16961, for VCA0956 and E. coli K12-MG1655 for yhjH, respectively). Introduced NheI and PstI restriction sites were used for cloning the products into pBAD42. A fragment containing the repressor encoding gene araC, the corresponding inducible promoter region, and gfp, VCA0956, or yhjH was then released by NsiI and PstI restriction. The fragment was gel purified and ligated into vector pME6041 digested with PstI, resulting in plasmids pARA-gfp, pARA-VCA0956, and pARA-yhjH. Orientation of the fragment relative to the transcriptional terminator flanking the multiple cloning site of pME6041 was ensured by restriction analysis. A second inducible system was constructed based on the lacIQ1-Ptac promoter system. A fragment containing the lacIQ gene and the tac promoter was amplified from the vector pMAL-c2 (New England Biolabs), introducing BamHI and PstI restriction sites at the 5′- and 3′-ends, respectively.

Additionally, the primer design yielded a mutation in the −35 region of the lacIQ promoter in order to obtain a higher expression of the repressor and, thus, a tighter repression of the system. The fragment was digested with BamHI and PstI, and ligated into the broad host range-vector pME6041 that was treated with the same enzymes resulting in vector pLacTac. Genes to be cloned into this vector were amplified by PCR using chromosomal DNA of the corresponding microorganism, introducing PstI and EcoRI restriction sites at the 5′- and 3′-ends, respectively. Plasmids were introduced by electroporation.

Functionality of both inducible systems was tested in S. oneidensis MR-1 wild type by induction of gfp in planktonic cultures. Chromosomal insertion of a lacIQ1-Plac-gfp fusion was carried out using a Tn7 delivery system by four-parental mating using pSM2360 as delivery plasmid as described for S. oneidensis.

RNA extraction and RT-PCR. S. oneidensis MR-1 cells were grown in 50 ml MM for 7, 24, and 40 hours, respectively, before 25 ml culture were centrifuged for 5 min at 4° C. and 4000×g. The cell pellet was resuspended in 1 ml ice cold AE buffer (20 mM sodium acetate, 1 mM EDTA, pH 5.2) and centrifuged again for 5 min at 4° C. and 15000×g. After resuspension of the pellet in 500 μl ice-cold AE buffer, 900 μl of phenol-chloroform-isoamyl alcohol (25:24:1) preheated to 60° C. and 10 μl of a 25% (w/v) SDS solution were added and incubated for 10 min at 60° C. Following centrifugation at 4° C. and 15000×g, the aqueous phase was transferred into a fresh tube, 62.5 μl of a 2M sodium acetate solution (pH 5.2) and 500 μl phenol-chloroform-isoamylalcohol were added, followed by another centrifugation. This step was repeated until no interphase was visible. RNA was precipitated by addition of 2.5 volumes ethanol (96% v/v), incubation for 30 min at −80° C., and centrifugation at 4° C. and 15000×g. Following two washing steps with 75% (v/v) ethanol, the RNA was dried at room temperature. Prior to the reverse transcriptase (RT) reaction the RNA was subjected to a DNA digest. The RNA sediment was resuspended in 400 μl water and 50 μl DNase buffer (40 mM Tris-HCl, pH 8; 10 mM NaCl; 6 mM MgCl₂; 10 mM CaCl₂), and 5 μl DNasel (10 U/μl) was added. The reaction was incubated for 1 h at 37° C.

Subsequently, the RNA was precipitated as described above; this step was repeated three times. The RT reaction was carried out using the SuperScriptIII Kit (Invitrogen, Carlsbad, Calif.) according to the manufacturer's instructions at 25° C. for 5 min, 1 h at 50° C. and 15 min 70° C. using 1 μg RNA in 20 μl final volume. The product was used in a PCR reaction using suitable primers, and RNA without RT treatment was used as a negative control.

Biofilm cultivation and image acquisition. Biofilms were cultivated at 30° C. in LM medium in three-channel flow cells with individual channel dimensions of 1×4×40 mm. Microscope cover slips (Fisher Scientific, Pittsburgh, Pa.) were used as colonization surface, glued with silicone (GE Sealants & Adhesives, Hunterville, N.C.) onto the channels, and left to dry for 24 h at room temperature prior to use. Assembly, sterilization, and inoculation of the flow system was carried out essentially as described. Experiments were carried out in triplicates in at least two independent experiments.

For switching medium, the flow was arrested briefly, and the medium was exchanged in the bubble trap and the upstream tubing. This process took no longer than 1 minute, and control channels, where the medium flow was stopped in parallel without changing the medium, ensured that the observed effects were not due to that short arrest. Confocal laser scanning microscopy (CLSM) was performed at defined spots close to the inflow before and after the treatment. Stop-of-flow-induced detachment was carried out as described earlier. Briefly, the medium flow was arrested for 15 min, and subsequently resumed for the same amount of time. CLSM images were taken immediately before the stop-of-flow and after 15 minutes of flow.

Microscopic visualization and image acquisition of biofilms was conducted at the Stanford Biofilm Research Center using an upright Zeiss LSM510 Confocal Laser Scanning Microscope (Carl Zeiss, Jena, Germany) equipped with the following objectives: 10×/0.3 Plan-Neofluar, 20×/0.5W Achroplan, and 40×/1.2W C-Apochromat. For displaying biofilm images, CLSM images were processed using the IMARIS software package (Bitplane AG, Zürich, Switzerland) and Adobe Photoshop. Biofilm parameters, such as biomass, and average biofilm thickness were quantified with the program COMSTAT.

For complementation studies, biofilm experiments were carried out in 96-well microtiter plate assays using crystal violet. Strains were allowed to grow for 16 h prior to processing and spectrophotometrical quantification at 570 nm using a VERSAmax tunable microplate reader (Molecular Devices, CA).

Extraction and quantification of c-di-GMP from Shewanella oneidensis. Cells were grown at 30° C. in LM medium supplemented with 40 mM lactate and 0.2% L-arabinose, then centrifuged and washed with phosphate buffered saline (PBS). The pellets were then resuspended in PBS. C-di-GMP was extracted by heat and ethanol, in triplicate. Cells were heated at 100° C. for five minutes, and then ethanol was added to a final concentration of 65%. Samples were centrifuged, the supernatant retained, and the extraction was repeated. Combined supernatants were dried using a Speed-Vac, then stored for subsequent LC-MS analysis of the c-di-GMP.

For LC-MS analysis, dried samples were resuspended in 50 μl of 10 mM ammonium acetate buffer, vortexed, ultrasonicated, centrifuged, and the supernatant retained. This was repeated and the supernatants were combined. Samples were analyzed at the Stanford Mass Spectrometry Facility. An Agilent 1100 HPLC system equipped with an autosampler and degasser was used for solvent delivery and sample introduction. Samples were injected (10 μl or 20 μl) into a reverse phase C18 Targa column (2.1×40 mm, 3). The column was eluted at 30° C. at a flow rate of 0.5 mL/min with the following gradient: 0-0.3 min, 0% B; 1.5 min., 90% B; 1.6-2.0 min., 0% B; 3.0 min, 90% B; 4.0-8.0 min, 0% B (A: 20 mM ammonium acetate; B: acetonitrile).

The Quattro Premier (MicroMassWaters) triple quadrupole mass spectrometer equipped with electrospray ion source was used for peak detection. The collision energy was 30 eV and the cone voltage was 35 V. The c-di-GMP was detected in the multiple reaction monitoring mode (MRM) with the following transitions: 691.1_{—}151.9, 691.1_{—}248.0, 691.1_{—}539.8.

C-di-GMP was synthesized and used as aqueous solution in 10 mM ammonium acetate as an external authentic standard. Calibration curve was plotted for concentrations of 0, 31.25, 62.5, 125.0, 250.0, 500.0 fmol/μl in triplicate; the graph was linear in this range, with a correlation coefficient, R2, of >0.99. The limit of detection of this method was 10-15 fmols.

### Results

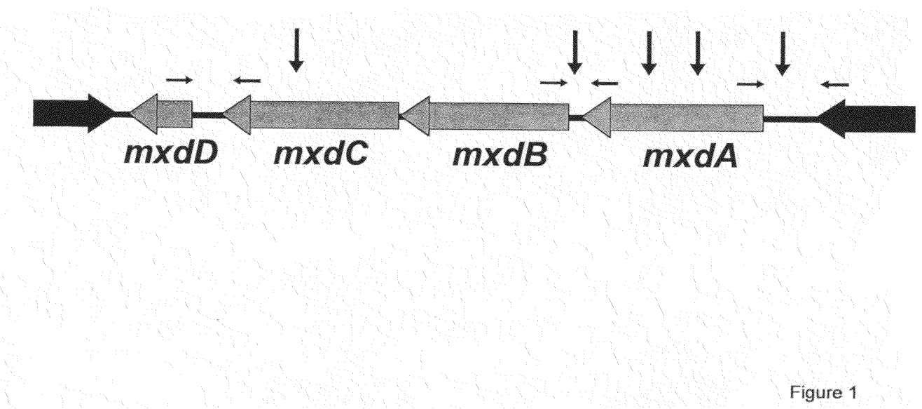

Identification of an EPS biosynthesis operon in S. oneidensis. In order to elucidate the connection between attachment and detachment, we focused on components controlling attachment of S. oneidensis cells to biofilms. Based on the S. oneidensis genome sequence, no genes encoding enzymes for EPS biosynthesis with similarity to those in Pseudomonas aeruginosa, E. coli, or Vibrio cholerae are obvious. In a genetic analysis using Tn5 mutagenesis coupled to a 96-well-based screen, we previously identified several mutants defective in biofilm formation. Among the mutants isolated were five with transposon insertions mapping to five independent positions of the gene cluster SO4180-4177, which we subsequently named, mxdA-D (for ‘biofilm matrix deficient’) because of the mutants' biofilm phenotype (see below). Two insertions were found in mxdA, one in the promoter region of mxdA, one in the intergenic region between mxdA and mxdB, and one insertion in mxdC (FIG. 1).

Analysis of the amino acid sequences of these orfs revealed that MxdA is predicted to be a 462 amino acid protein containing a C-terminal region with weak homology to a GGDEF domain. MxdB is predicted to be a membrane-associated 403 amino acid protein with homology to glycosyl transferases of the family GT 2 type, MxdC is predicted to be a 351 amino acid membrane-associated protein with homology to efflux pump proteins. MxdD is oriented in the same direction as the previous genes, and is predicted to be a 118 amino acid membrane-associated protein without homology to any known protein. The MxdB amino acid sequence was 25% identical and 42% similar to that of AcsAB, the cellulose synthase of G. xylinus, over a range of 192 amino acids. The orientation and sequences of the mxd genes are highly similar and homologous, respectively, only to the Vibrio parahaemolyticus RIMD genes VPA0392-94.

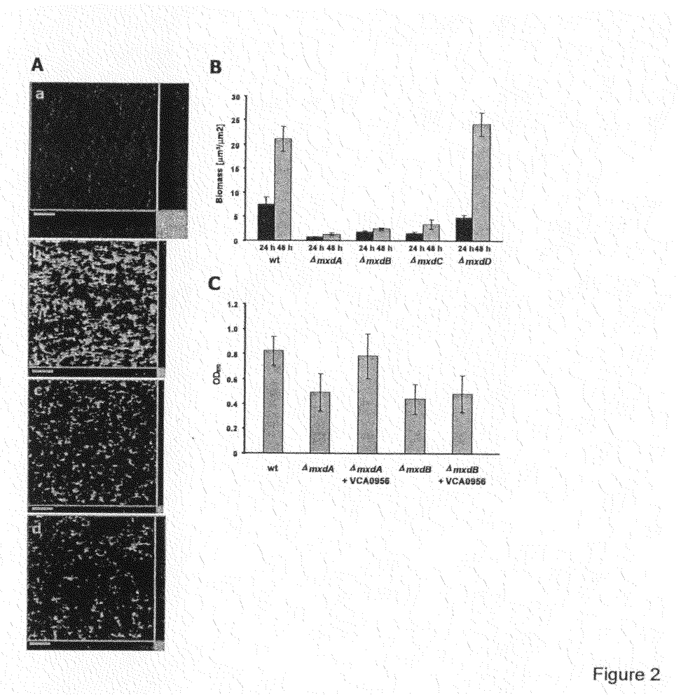

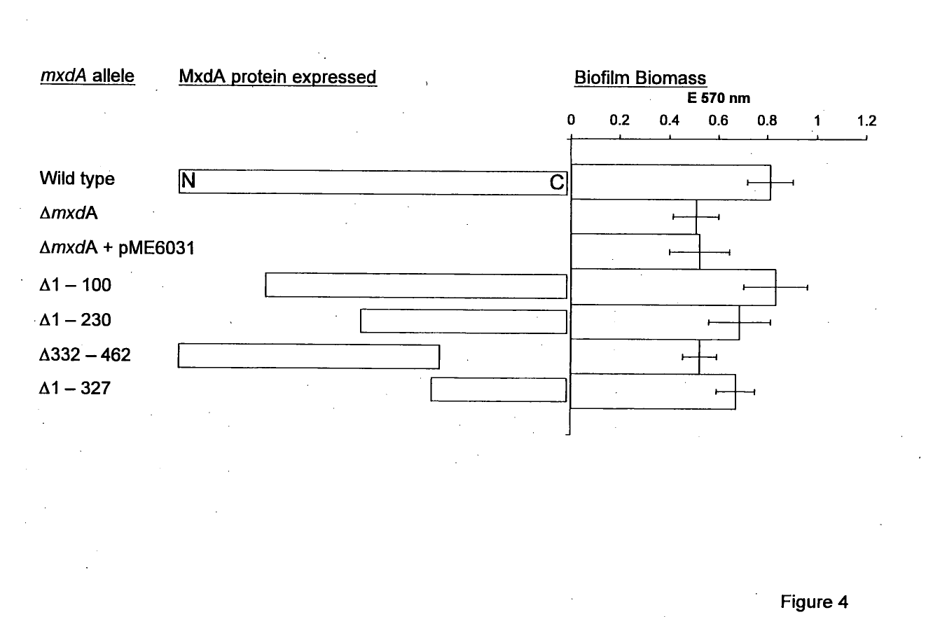

In order to examine the function of these genes in biofilm formation, we constructed in frame deletion mutants of all genes, and analyzed their phenotype in biofilms grown under hydrodynamic conditions. As FIG. 2a displays, deletion mutants of mxdA, mxdB, and mxdC exhibited strong defects in biofilm formation, and were severely impacted in developing a three-dimensional architecture. The initial adhesion of these mutants appeared to be similar to wild type. The mutant biofilms were arrested at the stage of a cell monolayer or few cell layers, and did not seem to progress from this stage even after 48 h incubation (FIG. 2A). The most severe phenotype was visible for the ΔmxdA mutant (FIG. 2A). Quantification of biofilm biomass revealed that biofilms of ΔmxdA, ΔmxdB, and ΔmxdC had between 84 and 94% less biomass than wild type biofilms (FIG. 2B) after 48 h. Deletion mutant ΔmxdD showed a delayed phenotype but progressed to a wild type-like architecture after 48 h. The in-frame deletion of mxdA but not of mxdB could be complemented by VCA0956, a known c-di-GMP-forming diguanylate cyclase (FIG. 2C). The wild type biofilm phenotype of both mutants could be restored by wild type gene expression in trans (FIG. 4) or by ‘knock-in’ gene reconstructions with the wild type alleles. These data, together with the sequence analysis suggested that the mxd genes might encode for a gene cluster essential for biofilm matrix formation in S. oneidensis.

The transcriptional organization of the mxdA-D genes was examined using reverse transcriptase (RT) PCR. When RNA prepared from cells grown to late exponential phase in a lactate mineral medium (see Material and Methods) was used as template, RT-PCR products were obtained for primer pairs probing for a contiguous mRNA between mxdA and mxdB, and mxdB and mxdC (FIG. 1). The reading frames of mxdB and mxdC are overlapping by one base and were therefore not probed. No RT-PCR product was observed from the primer pair combination SO4181 and mxdA. Preliminary transcriptional analysis revealed that mxdA-D mRNA is present in cells in late exponential and stationary, but not in early and mid exponential growth phase.

The role of c-di-GMP in biofilm formation and detachment. The finding of a potential GGDEF protein (MxdA) in the vicinity of a putative glycosyl transferase (MxdB) was intriguing: we speculated that in S. oneidensis the activity of an EPS synthase, such as MxdB, might also be regulated by c-di-GMP, which is synthesized by GGDEF domain proteins, conceivably such as MxdA. To examine this hypothesis, we tested whether the biofilm phenotype of ΔmxdA could be rescued by expressing VCA0956 in trans from an IPTG-inducible promoter. VCA0956 was previously shown to have c-di-GMP-forming diguanylate 1 cyclase activity. As evident from FIG. 2C, ectopic expression of this GGDEF protein complemented DmxdA to wild type level. However, VCA0956 did not complement a ΔmxdB mutation, suggesting that the diguanylate cyclase activity or any other activity associated with VCA0956 is not sufficient for rescue of the biofilm phenotype.

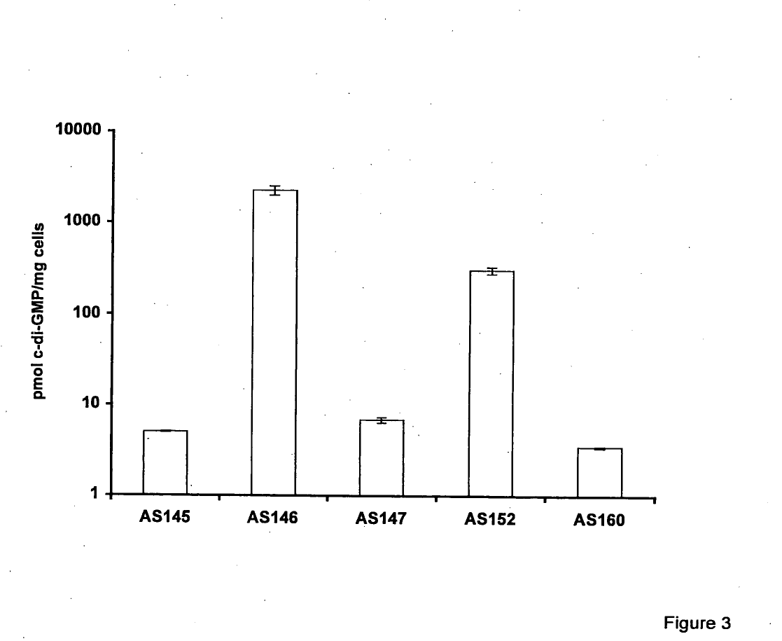

We then tested whether MxdA might encode a diguanylate cyclase by examining the intracellular level of c-di-GMP by LC-MS. LM-grown cells of strain AS152, which were induced with arabinose, were harvested, c-di-GMP was extracted and quantified by LC MS analysis as described in Material and Methods. Authentic c-di-GMP was used as a standard. As FIG. 3 shows, S. oneidensis strains carrying over-expressed VCA0956 (AS146) or mxdA (AS152) contained intracellular c-di-GMP levels that were several hundred-fold higher than in empty vector carrying wild type control strain (AS167) or the yhjH-over-expressing strain (AS145) (see below).

A truncation analysis of mxdA was conducted and revealed that the region with the most critical function, based on rescue of the mxdA biofilm phenotype, is a C-terminal region containing a NVDEF sequence at positions 357-361, which appears to resemble a modified GGDEF domain. Truncation of the first 230 amino acids resulted in intermediate complementation of the mxdA biofilm phenotype in a 96 well titer plate (FIG. 4). However, a mxdA allele where only the C-terminal NVDEF domain was deleted (Δ332-462) was unable to rescue the mutant phenotype. On the other hand, expression of the NVDEF domain alone was sufficient to partially confer rescue (FIG. 4). These data strongly suggest that MxdA can function as a diguanylate cyclase with an essential, modified GGDEF-like NVDEF domain. Moreover, these data provide the first indication that domains with a consensus motif significantly different from GGDEF, e.g., NVDEF can act as diguanylate cyclase in vivo.

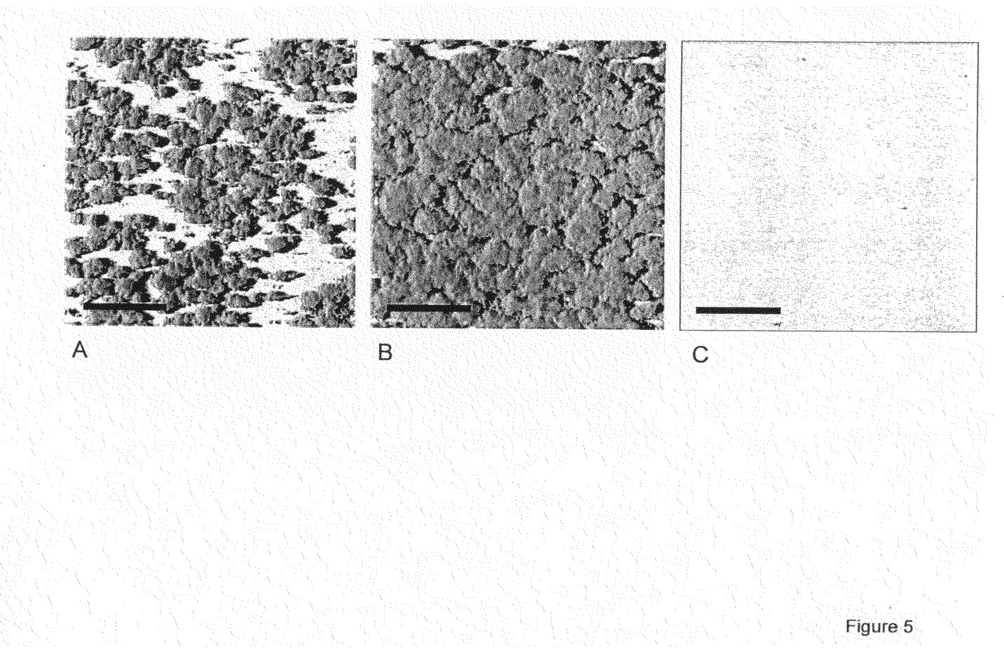

In order to test directly whether manipulation of the intracellular c-di-GMP concentration affects attachment and detachment of S. oneidensis, we constructed strains AS146, which carried VCA0956, and AS145, which carried yhjH, a S. typhimurium gene encoding an EAL-domain containing enzyme with c-1 di-GMP-hydrolyzing phosphodiesterase activity, under the control of inducible PBAD 2 promoter. To test these strains in biofilms, cells, including the empty vector-containing (pME6041) wild type strain AS147, were grown in LB medium containing 25 μg/ml kanamycin, diluted to an optical density of 0.01, and injected into the flow chambers to seed the glass surface. After 40 minutes incubation, flow was initiated with LM containing 25 μg/ml kanamycin and 0.2% arabinose as inducer. FIG. 5 displays representative images of 24 hr old biofilms. AS146 biofilms displayed a dramatically increased thickness compared to AS147. Strain AS146 was determined to have a nearly identical growth rate to AS147 in planktonic culture. It is therefore likely that the increased thickness observed in the AS146 biofilm was due to increased retention of cells in the biofilm. S. oneidensis wild type cells were observed to constantly shed a small fraction of cells during normal growth. Cessation or reduction of such shedding could retain cells in the biofilm as observed here.

In contrast, analysis of biofilms formed by cells of yhjH-expressing strain AS145 resulted in a dramatically different phenotype. After the 40 minutes incubation period following the injection of AS145 cells into the flow chamber, the cell density on the glass surface was indistinguishable from the control (AS147) or AS146. However, 30 minutes after starting flow of the arabinose-containing medium the adhering cells began to separate from the surface. As a result, practically no biofilm of AS145 had formed after 24 h under the inducing conditions (FIG. 5). This observation cannot be explained by the slightly lower growth rate of induced AS145 cells (ca. 70% of AS147 in planktonic culture). Rather, this unexpected finding indicates that induction of yhjH and by inference, of lower c-di-GMP level dramatically interfers with the maintenance of cell adhesion to the glass substratum. On the other hand, an increased level of cellular c-di-GMP leads to growth of a thick biofilm.

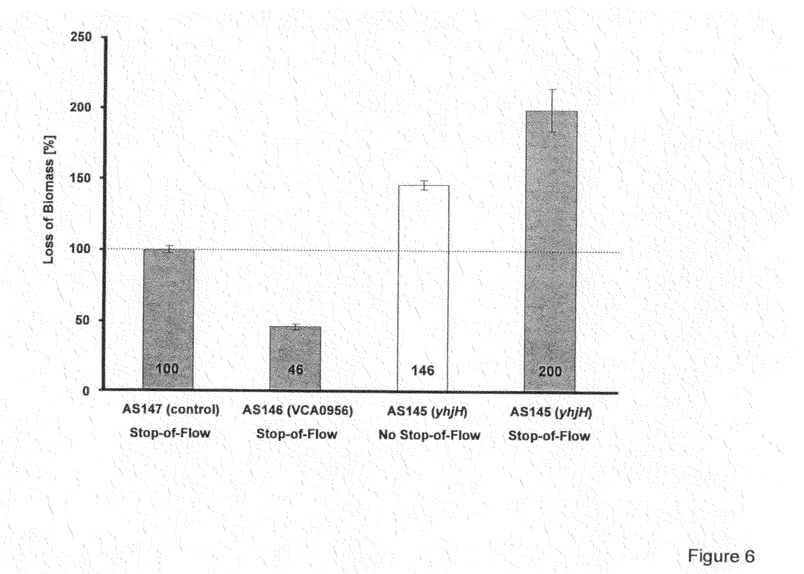

Our results collectively demonstrate a strong correlation between c-di-GMP level and cellular attachment to the biofilm matrix. This control might be mediated through the putative glycosyl transferase MxdB. Lowering of cellular c-di-GMP by over-expression of an EAL-domain protein encoding gene may induce detachment of biofilms in the absence of an external environmental cue. In S. oneidensis biofilms, we had previously identified that a decrease in molecular oxygen concentration functions as an external signal for detachment. In order to test this prediction, we grew biofilms of AS145 and AS146 in the hydrodynamic flow chamber in the absence of the inducer arabinose. After 20 h of growth the flow-through medium was amended with 0.2% arabinose, and biofilms were monitored by CSLM (FIG. 6). In contrast to AS147 and AS146, 60 to 70 minutes after induction, cells of AS145 began to detach from the biofilms in the absence of a stop-of-flow. After 90 min. 18% of the biofilm biomass was lost and after 120 min 38%.

In contrast, wild type biofilms harboring the empty plasmid (AS147) and biofilms of AS146 continued to increase in thickness after induction. A subsequent stop-of flow treatment of AS146 biofilms induced detachment of only 50% of that of wild type while the same treatment released from AS145 biofilms twice as much biomass (FIG. 6). These observations demonstrate that not only attachment but also detachment is controlled by c-di-GMP. Furthermore, induction of a gene encoding an EAL-containing protein with c-di-GMP-hydrolyzing phosphodiesterase activity can by-pass the requirement for an external environmental cue as the physiological signal and induce detachment.

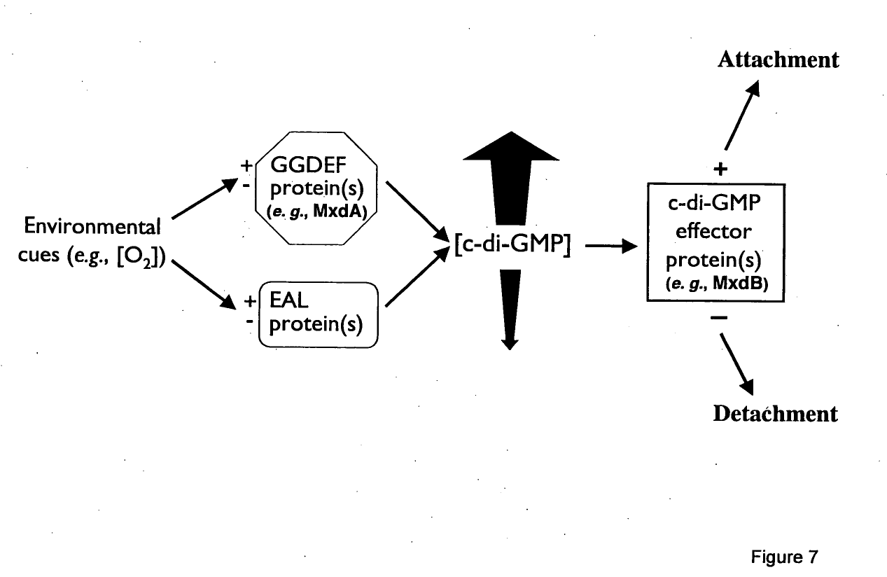

In this work, we examined the connection between attachment of biofilm cells to and detachment from the biofilm matrix in S. oneidensis, and provided data that both processes are linked by the mxd genes and by c-di-GMP. FIG. 7 summarizes the key components and their mode of interaction in controlling the transitioning of biofilm cells between attachment and detachment.

In genetic experiments we showed that a diguanylate cyclase, encoded by mxdA, is essential for matrix attachment and development of three-dimensional biofilm architecture (FIG. 2). The predicted MxdA gene product contains the essential C-terminal sequence NVDEF with weak homology to a GGDEF motif (FIG. 4); over-expression of mxdA increased the cellular c-di-GMP level (FIG. 3), and the ΔmxdA biofilm phenotype could be rescued by VCA0956, a gene encoding a GGDEF-domain containing protein with c-di-GMP-forming diguanylate cyclase activity (FIG. 2C). For these reasons, we concluded that MxdA contains diguanylate cyclase activity and increases when activated the in vivo cellular c-di-GMP level, which results in enhanced attachment and increased biofilm formation (FIG. 5). We postulate that this positive c-di-GMP signaling acts through MxdB, a putative membrane associated glycosyl transferase (FIG. 2C).

The mxdB gene product is essential for cell attachment to the matrix, but not for cell adhesion to the substratum surface (FIG. 2). Massive detachment, which was qualitatively and quantitatively indistinguishable from the oxygen starvation-induced detachment, could be induced by over-expression of yhjH encoding for an EAL-domain containing protein (FIG. 5, 6). This protein was previously shown to carry phosphodiesterase activity towards c-di-GMP. Thus, by simply modulating the intracellular c-di-GMP pool via over-expression of a diguanylate cyclase we were able to shift the state of biofilm cells from attachment or adhesion to detachment and vice versa under the same experimental conditions. In addition, this manipulation of the intracellular c-di-GMP pool allowed us to uncouple the physiological detachment response from the external environmental cue (FIG. 6).

We demonstrate that the mode of action of c-di-GMP in attachment and detachment is not primarily via control of gene regulation, as has been directly or indirectly postulated in several other systems, but by a direct (e.g., allosteric) control, for example of a polysaccharide synthesizing enzyme such as the MxdB (FIG. 7). An alternative mode of action of intracellular c-di-GMP could be in an indirect control of detachment and attachment by modulating cellular sensitivity to variation in local oxygen concentration, where low c-di-GMP renders cells more susceptible to fluctuations in oxygen. In light of these results, our previously identified dependence of the detachability of S. oneidensis biofilms on CRP, ArcA, and EtrA, can therefore be attributed to an indirect role of these transcriptional regulators. For example, they may be required for controlling the expression of the molecular detachment/attachment machinery or signal transduction components, as we had suggested.

All publications, patents, and patent applications cited in this specification are herein incorporated by reference as if each individual publication, patent, or patent application were specifically and individually indicated to be incorporated by reference.

The present invention has been described in terms of particular embodiments found or proposed by the inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in methods, structures, and compounds without affecting the biological action in kind or amount. All such modifications are intended to be included within the scope of the appended claims.

What is claimed is:

1. A method of inhibiting microbial biofilm maintenance or formation, the method comprising:
contacting microbes involved in said biofilm formation or maintenance with an agent that interferes with cyclic-di-GMP signaling.

2. The method according to claim 1, wherein said agent is an analog of cyclic-di-GMP.

3. The method according to claim 2, wherein said inhibitor has the structure: 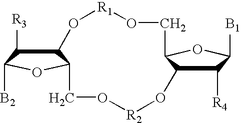
where B₁ and B₂ are independently selected from nitrogenous bases and derivatives thereof, including guanosine, inosine, adenine, guanine derivatives modified at the 6-position with S, N and O heteroatoms, 6-thioguanine, 2,6-diaminopurine, O6-alkyl guanine derivatives;
R₁ and R₂ are independently selected from PO₂; C═O; O—P═S (phosphorothioate); S═P═S (phosphorodithioate); O═P—BH₃ (boranophosphates); phosohoroamidite; O═P—CH₃ (methyl phosphonate); methane phosphonamidite; amide; methylene(methylimino); thioformacetal; dimethylene sulfone; sulfonamide; sulfonate; 5′N-sulfamate; sulfamide; 3′N-sulfamate; replacement of the entire phosphodiester with a guanidium or morpholino group;
R₃ and R₄ are independently selected from H, hydroxyl, ethers of lower alkyls; esters; CO₂H; thiols; phosphates, boronates lower alkyls, including methyl, ethyl, propyl, butyl, t-butyl.

4. The method according to claim 1, wherein said the biofilm inhibitor is a genetic sequence that interferes with cyclic-di-GMP synthesis or signaling.

5. The method according to claim 4, wherein said genetic sequence inhibits expression of an mdx operon gene or homolog thereof.

6. The method according to claim 1, wherein said biofilm is present in vitro.

7. The method according to claim 1, wherein said biofilm is present in vivo.

8. The method according to claim 7, further comprising administering a bactericidal agent in combination with said agent that interferes with cyclic-di-GMP signaling.

9. A method of screening for agents that inhibit biofilm maintenance of formation, the method comprising:
contacting a polypeptide or cell with a candidate agent suspected of interfering with cyclic-di-GMP signaling.

10. The method according to claim 9, wherein said cell is provided as a biofilm of tester bacteria; and the loss of biomass from the biofilm in response to the candidate agent is quantified.

11. The method according to claim 9, wherein said peptide comprises a GGDEF-like domain.

12. The method according to claim 11, wherein binding of said agent to said peptide is quantified.

13. The method according to claim 11, wherein the effect of said agent on diguanylate cyclase activity is quantified.

14. The method according to claim 11, wherein said peptide comprises at least a fragment of mdxA.

15. The method according to claim 14, wherein said fragment comprises the amino acid motif NVDEF.
